(19) United States
(12) Patent Application Publication
(10) Pub. No.: US 2011/0303909 A1
(43) Pub. Date: Dec. 15, 2011

(54) PLANAR CONJUGATED COMPOUNDS AND THEIR APPLICATIONS FOR ORGANIC ELECTRONICS

(75) Inventors: Zhikuan Chen, Singapore (SG); Qinde Liu, Singapore (SG); Samarendra P. Singh, Singapore (SG); Achmad Zen, Singapore (SG)

(73) Assignee: Agency for Science, Technology and Research, Connexis (CN)

(21) Appl. No.: 13/202,752

(22) PCT Filed: Feb. 20, 2009
(86) PCT No.: PCT/SG09/00061

§ 371 Date: Aug. 22, 2011

(51) Int. Cl. H01L 51/10; C07D 409/04; C07D 333/08

(52) U.S. Cl. 257/40; 438/99; 549/80; 549/59; 257/E51.004

(57) ABSTRACT

The invention relates to organic semiconducting materials, methods for their preparation and organic electronic devices incorporating the said organic semiconducting materials. The organic semiconductors contain a compound of formula (I)

Ar₁=(Qu)ₘ=Ar₂ (I)

where Qu is independently a substituted or unsubstituted, substantially planar 5 to 8 membered conjugated ring, and Ar₁ and Ar₂ each independently is a substituted or unsubstituted, substantially planar conjugated aromatic structure having from 5 to 50 carbon atoms. The compounds of formula may generally form an H-shaped molecular structure. The said organic semiconducting materials could be used as the active layers for organic electronic devices, e.g. thin film transistors, photovoltaic cells, photo detectors, light emitting diodes, memory cells, sensors etc.

### FIELD

The invention relates to organic semiconducting materials, their preparation methods, and their application in organic electronic devices.

### BACKGROUND

Organic molecules and polymers with a π-conjugated backbone may be applied in electronic devices, including optoelectronic devices, e.g. organic light emitting diodes, photovoltaic cells, thin film transistors, memory cells and sensors. Compared to inorganic semiconductors, organic semiconductors may be processed into large area and flexible devices with low cost. Organic materials that have been used as semiconductors include both sublimed and solution processed semiconductors, such as pentacene, copper phthalocyanines, hexadecafluorocopper phthalocyanines, fullerene based materials, naphthalene carbodiimide derivatives, oligothiophenes, polythiophenes, polyfluorene base copolymers, polytriarylamine based copolymers, poly(p-phenylene vinylene) (PPV) based copolymers, etc.

Recently, a number of low bandgap polymers have been developed, such as poly[cyclopentadithiophene] and derivatives thereof, which may be used as a light absorption layer for organic photovoltaic (OPV) applications. Some of the known organic semi-conductor materials have demonstrated either good charge mobility for thin film transistor (TFT) application or good power conversion efficiency for OPV application. For example see, Leufgen et al. Organic Electronics, 2008, 9(6), p. 1101-1106 and Doi et al. Chemistry of Materials, 2007, 19(22), p. 5230-5237, where n-tetrathiafulvalene derivatives, organic electronic elements and electronic apparatus containing the'organic elements are described; Tang at al. Synthetic Metals, 2005, 115(1), p. 100-104, where synthesis, structure and properties of a quinoid compound is described; and WO 2007/118799 and WO 2007/068618 which relate to quinoid systems as organic semiconductors, and organic semiconductors and their manufacture, respectively. However, there is a still a need to further develop organic polymers with π-conjugated backbones for use as semiconductors.

### SUMMARY

The invention relates to organic semiconducting materials, their preparation methods, and their application in organic electronic devices.

According. to one aspect of the present invention; there is a provided a compound of formula (I):

Ar₁=(Qu))ₘ=Ar₂ (I)

where

each Qu is independently a substituted or unsubstituted, substantially planar 5 to 8 membered conjugated ring, optionally fused to one or more aromatic moieties independently chosen, and may contain one or more heteroatoms independently chosen,

Ar₁ and Ar₂ each independently is a substituted or unsubstituted, substantially planar conjugated aromatic structure having from 5 to 50 carbon atoms, optionally having one or more heteroatoms independently chosen, and is optionally substituted by one or more substantially planar conjugated aromatic structures independently chosen, and

m is from 1 to 20.

In an embodiment of the present invention, there is provided a compound of formula (Ia)

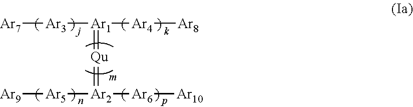

where

Qu, Ar₁, Ar₂ and m are as described above,

Ar₃, Ar₄, Ar₅, Ar₆, Ar₇, Ar₈, Ar₉ and Ar₁₀ each independently is a substituted or unsubstituted, substantially planar conjugated aromatic structure having from 5 to 50 carbon atoms, and optionally contains one or more heteroatoms independently chosen, and

j, k, n, p each, independently, is from 0 to 20.

According to another aspect of the present invention, there is provided a compound of formula (Ib):

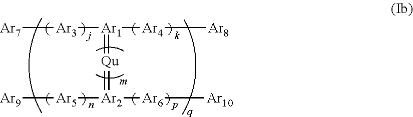

where

each Qu is independently a substituted or unsubstituted, substantially planar 5 to 8 membered conjugated ring; optionally fused to one or more aromatic moieties independently chosen, and may contain one or more heteroatoms independently chosen,

Ar₁, Ar₂, Ar₃, Ar₄, Ar₅, Ar₆, Ar₇, Ar₈, Ar₉ and Ar₁₀ each, independently, forms a substituted or unsubstituted, substantially planar conjugated aromatic structure having from 5 to 50 carbon atoms, and optionally contains one or more heteroatoms independently chosen,

m is from 1 to 20,

j, k, n, p each, independently, is from 0 to 20, and

q is 1 or more.

According to still another aspect of the invention, there is provided a compound of formula 1, 2, 3 or 4.

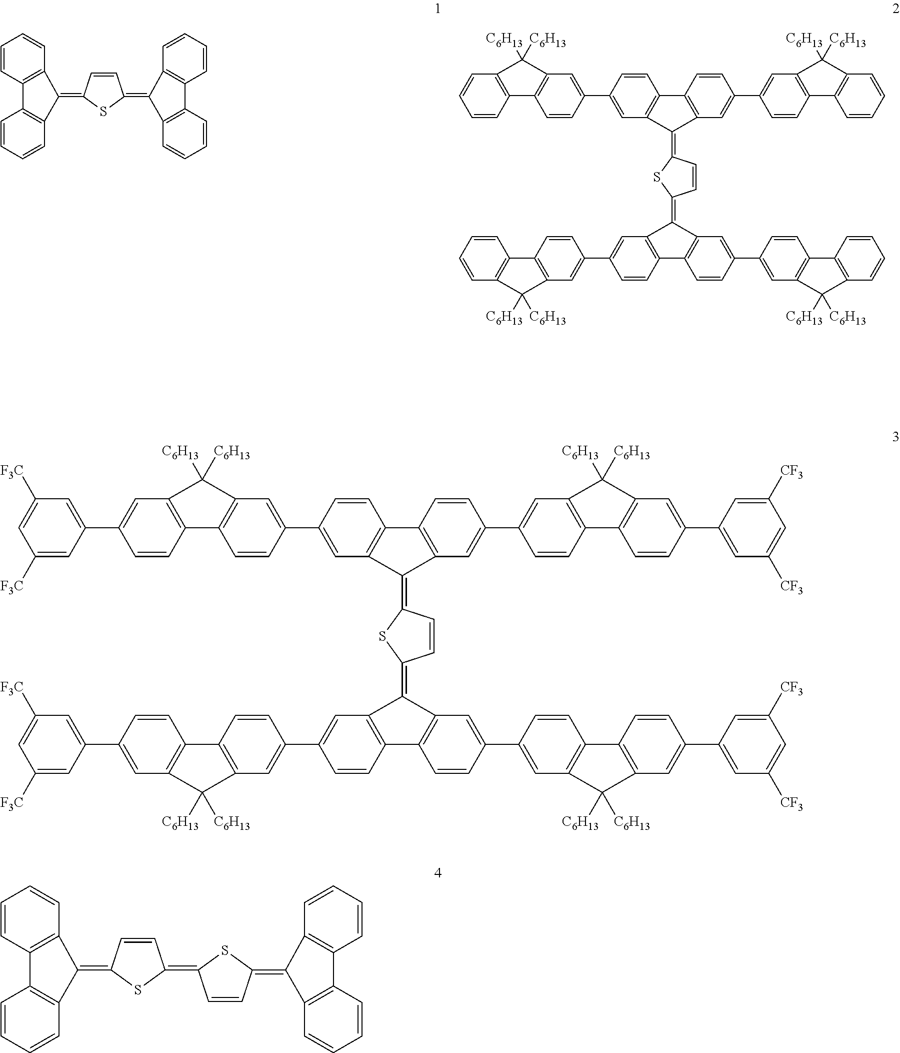

According to yet another aspect of the invention, there is provided a semiconductor device having a semi-conductor layer containing a compound of the invention as herein described.

According to yet a further aspect of the present invention, there is provided a use of a compound of formula (I), (Ia) or (Ib), as described above, in an organic electronic device.

According to yet a further aspect of the present invention, there is provided a device comprising a compound of formula (I), (Ia) or (Ib), as described above.

### BRIEF DESCRIPTION OF FIGURES

FIG. 1 shows the cyclic voltammogram (CV) of compound 1, recorded in dichloromethane with 0.1M tetrabutylammonium hexafluorophosphate as supporting electrolyte (scan rate of 50 mV/s) using a three electrode configuration consisting of a platinum wire working electrode, a gold counter electrode, and a Ag/AgCl in 3 M KCl reference electrode. The measured potentials were converted to saturated calomel electrode (SCE).

FIG. 2 displays schematically a structure of an organic thin film transistor device.

FIG. 3 shows the voltage applied on source and drain electrodes versus the current flow through source and drain electrodes (V_{ds}-I_{ds}) characteristic of compound 1 based OTFT on octadecyltrichlorosilane (OTS) treated p⁺-Si/SiO₂ substrate.

FIG. 4 shows the voltage applied on source and gate electrodes versus the current flow through source and drain electrodes (V_{gs}-I_{ds}) characteristic of compound 1 based OTFT on OTS treated p⁺-Si/SiO₂ substrate.

FIG. 5 displays the current-voltage characteristics of an OPV device with ITO/PEDOT:PSS/1/C₆₀/Al (black line) (where ITO=indium tin oxide; PEDOT=polyethylenedioxythiophene; PSS=polystyrene sulfonic acid) and ITO/PEDOT:PSS/1/1:C₆₀/C₆₀/Al (dotted line) upon illumination by AM1.5 solar simulator with an intensity of 50 mW/cm².

FIG. 6 displays the current-voltage characteristics of an OPV device with configuration of ITO/PEDOT:PSS/P3HT:Compound 3/Al (P3HT=polythiophene) upon illumination by AM1.5 solar simulator with an intensity of 50 mW/cm².

FIG. 7 displays the current-voltage characteristics of an OPV device with configuration of ITO/PEDOT:PSS/P3HT:Compound 3/Al annealed at 150° C. for 30 min., upon illumination by AM1.5 solar simulator with an intensity of 50 mW/cm².

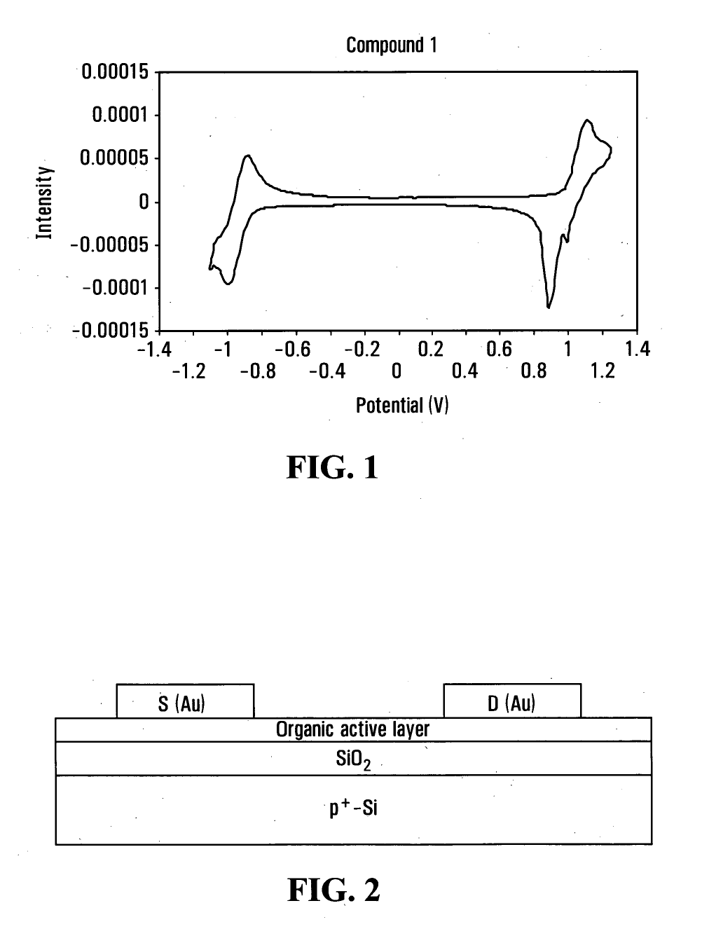

### DETAILED DESCRIPTION

Generally stated, organic semiconducting materials of the invention are composed of two conjugated molecular backbones, in which the two conjugated molecular backbones are linked by a substantially planar 5 to 8 membered conjugated ring, for example a quinoid-like group, as a bridge, as shown in formula (I) above. The resulting structure may generally-resemble an H-shaped molecular structure, for example. Organic semiconducting materials of the invention may be p-type materials or n-type materials and may be used as active layers for organic electronic devices, e.g. thin film transistors, photovoltaic cells, photo detectors, light emitting diodes, memory cells, or sensors. Organic semiconductor materials of the invention may be applied as active layers through solution process or vacuum deposition, for example.

As described above, the present invention provides a compound of formula (I)

Ar₁=(Qu)ₘ=Ar₂ (I)

where

each Qu is independently a substituted or unsubstituted, substantially planar 5 to 8 membered conjugated ring, optionally fused to one or more aromatic moieties independently chosen, and may contain one or more heteroatoms independently chosen,

Ar₁ and Ar₂ each independently is a substituted or unsubstituted, substantially planar conjugated aromatic structure having from 5 to 50 carbon atoms, optionally having one or more heteroatoms independently chosen, and is optionally substituted-by one or more substantially planar conjugated aromatic structures independently chosen, and

m is from 1, to 20.

In one embodiment there is provided a compound of formula (Ia)

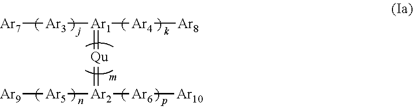

where

Qu, Ar₁, Ar₂ and m are as described above,

Ar₃, Ar₄, Ar₅, Ar₆, Ar₇, Ar₈, Ar₉ and Ar₁₀ each independently is a substituted or unsubstituted, substantially planar conjugated aromatic structure having from 5 to 50 carbon atoms, and optionally contains one or more heteroatoms independently chosen, and

j, k, n, p each, independently, is from 0 to 20.

For the purpose of illustration and without limitation, compounds of formula (I) or (Ia) may have a molecular weight (M.W.) range of from 300 to 300,000, and including any intermediate value or range therein.

The present invention also provides a compound of formula (Ib):

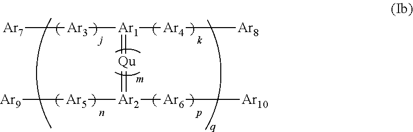

where

each Qu is independently a substituted or unsubstituted, substantially planar 5 to 8 membered conjugated ring, optionally fused to one or more aromatic moieties independently chosen, and may contain one or more heteroatoms independently chosen,

Ar₁, Ar₂, Ar₃, Ar₄, Ar₅, Ar₆, Ar₇, Ar₈, Ar₉ and Ar₁₀ each, independently, forms a substituted or unsubstituted, substantially planar conjugated aromatic structure having from 5 to 50 carbon atoms, and optionally contains one or more heteroatoms independently chosen,

m is from 1 to 20,

j, k, n, p each, independently, is from 0 to 20, and

q is 1 or more.

For the purpose of illustration and without limitation, compounds of formula (Ib) may have a molecular weight (M.W.) range of from 300 to 1,000,000, and including any intermediate value or range therein.

For the purpose of illustration and without limitation, q in the compounds of formula (Ib) may range from 1 to 100, and including any intermediate value or range therein.

In yet another embodiment of the invention, there is provided a compound as described above having a low bandgap (for example, 2 eV).

In another embodiment of the invention, there is provided a compound of formula (I), (Ia) or (Ib) as described above, wherein any one of Ar₁, Ar₂, Ar₃, Ar₄, Ar₅, Ar₆, Ar₇, Ar₈, Ar₉ and Ar₁₀ forms a rigid, substituted or unsubstituted, substantially planar conjugated aromatic structure.

In a still another embodiment of the invention, there is provided a compound of formula (I), (Ia) or (Ib) as described above, wherein any one of Ar₁, Ar₂, Ar₃, Ar₄, Ar₅, Ar₆, Ar₇, Ar₈, Ar₉ and Ar₁₀ forms a bridged, substituted or unsubstituted, substantially planar conjugated aromatic structure.

In yet a further embodiment of the invention, the substitutions on Ar₁ to Ar₁₀, may be one or more electron donating groups, electron withdrawing groups or a combination thereof.

The compound of formula (I), (Ia) or (Ib) as defined above may form an H-shaped structure. Without being bound to a theory, it is believed that a compound of the invention having an H-shaped structure in a planar configuration may facilitate the formation of 3-dimensional ordered structures in a film. The molecule may be fully conjugated along both backbones and also the bridge of the H-shaped structure, which may be beneficial for charge migration and/or transport from one backbone to another. The compounds of the invention may also possess lower highest-occupied-molecular orbital (HOMO) and lowest-unoccupied-molecular orbital (LUMO) energy levels, and possibly much. lower LUMO energy levels than conventional p-type organic semiconductors. The lower HOMO energy levels may enhance the stability of the compounds applied in organic electronic devices and the lower LUMO energy levels may lead to lower bandgap (difference in energy between LUMO and HOMO) and may, therefore, also be beneficial for organic photovoltaic (OPV) devices with high open-circuit voltage (V₀). For the purpose of illustration and without limitation, a low HOMO may be <−5.1 eV, while a low LUMO may be <−3.0 eV.

Some of the features that may, therefore, be present in the compounds of the invention may include one or more of: (1) H-shape planar conjugated structure, (2) low HOMO energy levels, (3) even lower LUMO energy levels and (4) low bandgap. One or more of the above mentioned properties can be helpful to improve the charge mobility for TFT, light absorption efficiency, V_{oc} and power conversion efficiency for OPVs. These properties may be achieved through selection of bridge and backbone moieties, and substituents thereof.

In a compound of formula (I), (Ia) or (Ib), =Qu= represents a bridge between substantially parallel planar conjugated structures represented by Ar₁ and Ar₂. In the compound of formula (I), Qu is joined by a pair of double bonds to each of Ar₁ and Ar₂, respectively. Further, Qu may contain one or more substituents, such as a fused aromatic moiety or a heteroatom, or both. In one embodiment, Qu may form a quinoid-like moiety, such as

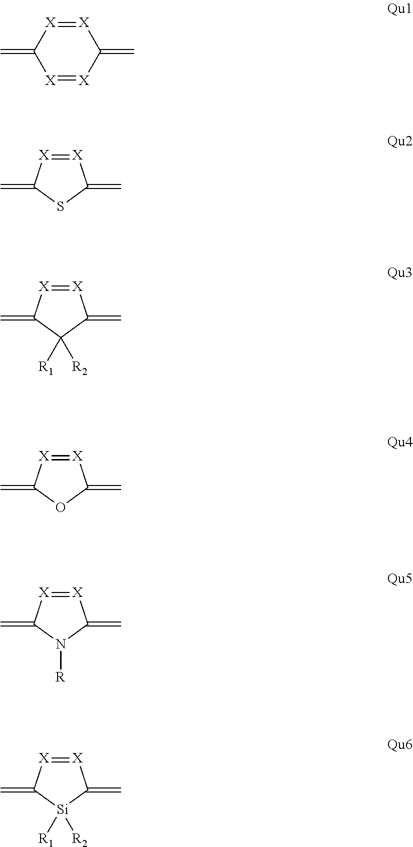

where each X is, independently, C, CH, N or P, and wherein each quinoid-like moiety is optionally independently substituted by one or more alkyl, alkoxyl or aryl substitutents, or may be fused to one or more aromatic moieties.

Further, each of Ar₁ and Ar₂ in the compound of formula (I), (Ia) or (Ib), as described above, generally forms a substantially planar conjugated aromatic structure. Without being bound to any one theory, it is believed that the planar structure may allow for better interaction of the n-orbitals within each of Ar₁ and Ar₂. This may also be achieved by forming a rigid conjugated aromatic structure, for example, without limitation and for the purposes of illustration, by means of bridging within each of the Ar₁ and Ar₂, such as, in an arylene group as shown in compound 1, shown above. Another way of achieving this may be through substitution of either or both of the groups. Substituents may be chosen to limit .the rotational freedom of Ar₁ and Ar₂ groups. The substituents may be present on any one of Qu, Ar₁ or Ar₂. The above bridging or substitution may lead to reduced rotational freedom and may lead to a planar conjugated aromatic structure, and hence, may permit improved interaction of the n-orbitals. Selection of substituents may also, for example, enhance the processability for device fabrication.

Ar₁ and Ar₂ are can each independently be, without limitation and for the purposes of illustration, an arylene group, an arylene vinylene group, an arylene ethynylene group, a heteroarylene group, a heteroarylene vinylene group or a heteroarylene ethynylene group, that can form a double bond with central Qu. Ar₁ and Ar₂ may have 5 to 50 nucleus carbon atoms which may be substituted, and may contain one or more O, S, N, Si, or P hetero atoms.

The Ar₁=Qu=Ar₂ quinoid structure may also be substituted by one or more further aromatic groups, such as in a compound of Formula (Ia), as defined above. Each Ar₃, Ar₄, Ar₅, Ar₆, Ar₇, Ar₈, Ar₉, and Ar₁₀ in a compound of Formula (Ia) are independently chosen and may be an arylene group, an arylene vinylene group, an arylene ethynylene group, a heteroarylene group, a heteroarylene vinylene group or a heteroarylene ethynylene group, having 5 to 50 nucleus carbon atoms, which may be substituted, and may contain one or more O, S, N, Si, or P heteroatoms.

The repetition number j, k, n, p is an integer of 0 to 20, while the repetition number m is an integer of 1 to 20.

The substitutions on Qu or any one of Ar₁ to Ar₁₀ can include, for the purposes of illustrations and without limitation, one or more functional groups, such as, halogen, a hydroxyl group, a carbonyl group, an amine group, a thiol group, a nitro group, a nitrile group or a cyano group, or can be a substituted or unsubstituted alkyl chain, alkoxy chain, alkylthio chain, alkylamino chain, alkenyl chain, alkynyl chain, aryl, heteroaryl, arylamino, heteroaryl amino, aryloxy, heteroaryloxy, arylthio, heteroarylthio, aralkyl, heteroarylalkyl, alkylsilyl or arylgermyl. Each of these may further be substituted with one or more functional groups.

For the purposes of illustration, where Qu is a quinoid-like moiety, it may be selected from the following listed structures, or may be a heterocyclic quinoid-like moiety that contains S, N, O, P, Si atoms. In the structures listed below, each X could independently be CH, N, or P, and may be substituted by alkyl, alkoxyl or aryl groups.

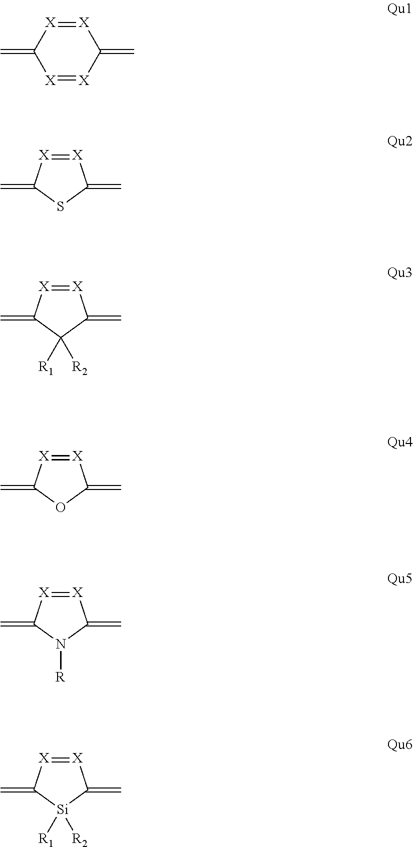

The Ar₃, Ar₄, Ar₅, Ar₆, Ar₇, Ar₈, Ar₉, Ar₁₀ can be divided into two groups, the connecting group, which include Ar₃-Ar₆, and the end-cap group, which includes Ar₇-Ar₁₀. One or more substitutions in the connecting group or one or more substitutions in the end-cap group may be electron donating groups. Electron donating groups may be, for example and without limitation, thiophenyl, triarylamino, or carbazolyl groups, or other electron donating aryl, arylene, or arylene vinylene groups. Electron donating groups may increase the hole transporting ability of the materials.

One or more substitutions in the connecting group or one or more substitutions in the end-cap group may be electron donating groups. Electron donating groups may be, for example and without limitation, a group (a1) to (a33), identified below, or may be one or more halo group, cyano group, nitro group, carbonyl group, thionyl group, sulphonyl group, or perfluoroalkyl group. Electron donating groups may increase the stability of the materials.

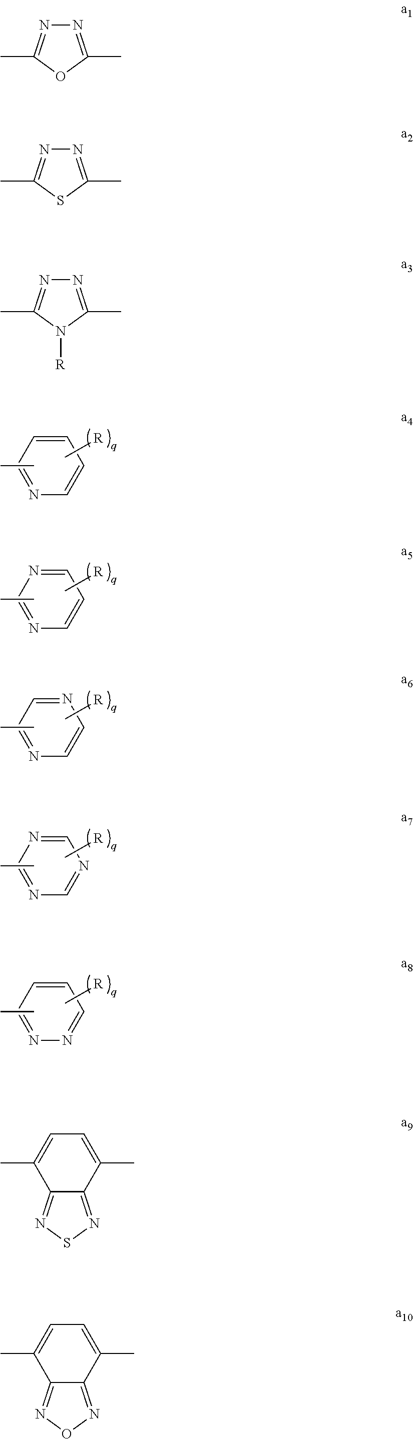

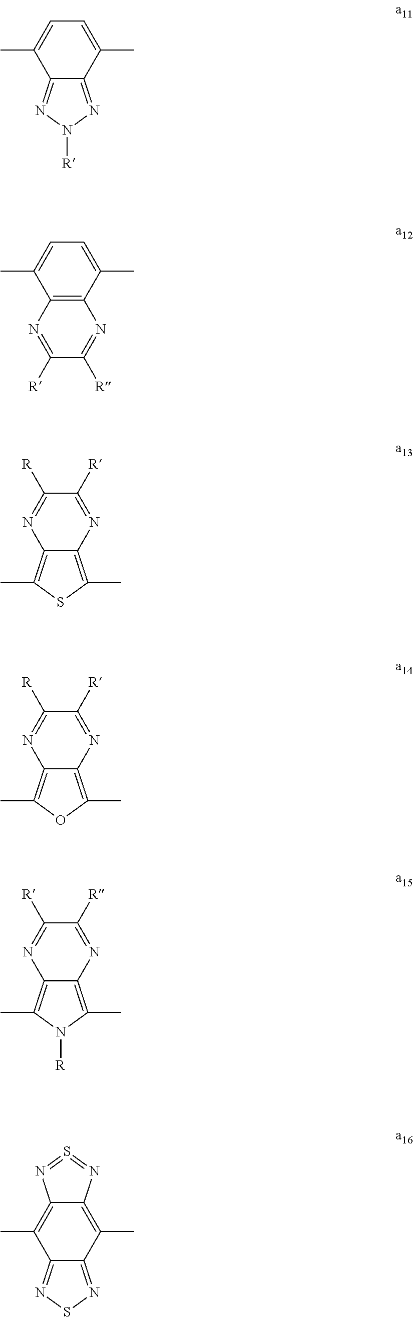

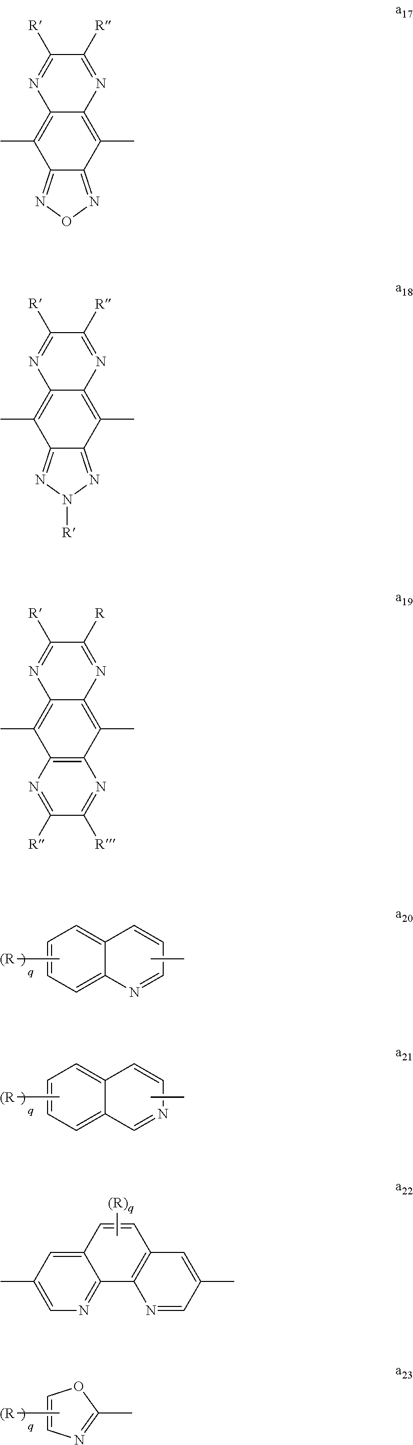

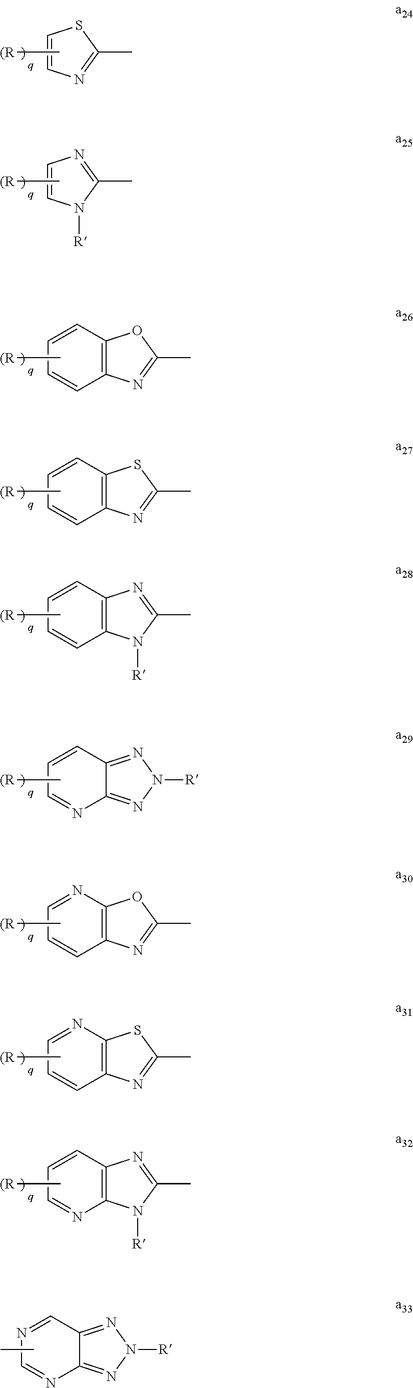

The term ‘semiconductor’ as described herein is generally understood as being a material that has an electrical conductivity between those of a conductor and an insulator.

The symbol “/” means an interface between the two materials and the symbol “:” when separating two materials means that both are in the same layer.

The term ‘p-type’ refers to a semiconductor that prefers to conduct holes.

The term ‘n-type’ refers to a semiconductor that prefers to conduct electrons.

The term ‘material’ generally refers to a substance, for example, a semiconductor layer containing a compound of the invention.

The term ‘conjugation’ generally refers to a system of atoms covalently bonded with alternating single and multiple, for example, double bonds. The double bond may be replaced by an atom having a lone pair of electrons, such as a heteroatom in thiophene. This system may provide in ,general delocalization of the electrons across all of the adjacent parallel aligned p-orbitals of the atoms.

The term ‘substantially planar conjugated ring or structure’ refers to a conjugated ring or structure that would allow favourable interaction of the n-orbitals, as has been described above. Thus, a compound having a substantially planar conjugated ring or structure would have a low dihedral angle within the conjugated system, for example.

The term ‘fused aromatic moiety’ as described herein, refers to, for example, naphthylene, anthrenylene, indenylene, azulenylene or phenanthrenylene. In the fused aromatic moiety, the examples described above may have two atoms that form part of both Qu and the fused aromatic moiety.

The term ‘heteroatom’ as described herein refers to an atom, such as, O, S, N, Si or P, for example.

The alkyl chain as used anywhere herein unless otherwise specified, may have from 1 to 30 carbon atoms. In some embodiments, the alkyl chain may have, for example, from 1 to 18 carbon atoms. In other embodiments, the alkyl chain may be, for example a C₁₋₆ alkyl, and may be without limitation, any straight or branched alkyl, for example, methyl, ethyl, n-propyl, i-propyl, sec-propyl, n-butyl, i butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, 1-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl or 3-methylpentyl. The C₁₋₆ alkyl group may contain one or more substituents and may further be, for example, and without limitation, interrupted by one or more heteroatoms which are independently nitrogen, sulfur or oxygen.

The alkoxy, alkylthio, alkylamino or other heteroalkyl groups as used anywhere herein unless other specified may have from 1 to 30 carbon atoms. In some embodiments, the alkyl chain may have, for example, from 1 to 18 carbons atoms. For the purpose of illustration, the carbon atom chains of the alkoxy, alkylthio, alkylamino or other heteroalkyl groups is the same as the alkyl chain described herein.

The alkenyl group as used anywhere herein unless otherwise specified, may have from 1 to 30 carbon atoms. In some embodiments the alkyl chain may have, for example from 1 to 18 carbon atoms. In other embodiments, the alkenyl chain may be, for example, a C₁₋₆ alkenyl, and may be without limitation, any straight or branched alkenyl, for example, vinyl, allyl, isopropenyl, 1-propene-2-yl, 1-butene-1-yl, 1-butene-2-yl, 1-butene-3-yl, 2-butene-1-yl, 2-butene-2-yl, 1-propene-2-methyl-1-yl, 2-propene-2-methyl-1-yl, 1-pentene-1-yl, 2-pentene-1-yl, 3-pentene-1-yl, 4-pentene-1-yl, 1-butene-3-methyl-1-yl, 2-butene-3-methyl-1-yl, 3-butene-3-methyl-1-yl, 1-butene-2-methyl-1-yl, 2-butene-2-methyl-1-yl, 3-butene-2-methyl-1-yl, 2-pentene-2-yl, 2-pentene-3-yl, 2-pentene-4-yl, 1-pentene-4-yl, 1-butene-3-methyl-2-yl, 1-butene-2-methyl-3yl, 1-hexene-1-yl, 2-hexene-1-yl, 3-hexene-1-yl, 4-hexene-1-yl, 5-hexene-1-yl, 1-hexene-2-yl, 2-hexene-2-yl, 3-hexene-2-yl, 4-hexene-2-yl, 5-hexene-2-yl, 1-hexene-3-yl, 2-hexene-3-yl, 3-hexene-3-yl, 2-hexene-4-yl, 1-hexene-4-yl, 1-pentene-4-methyl-1-yl, 2-pentene-4-methyl-1-yl, 3-pentene-4-methyl-1-yl, 1-pentene-2-methyl-5-yl, 1-pentene-4-methyl-2-yl, 2-pentene-4-methyl-2-yl, 2-pentene-2-methyl-4-yl, 1-pentene-2-methyl-4-yl, 1-pentene-4-methyl-3-yl, 2-pentene-4-methyl-3-yl, 2-pentene-2-methyl-3-yl, 1-pentene-2-methyl-3-yl, 1-butene-3,3-dimethyl-1-yl, 1-butene-2,3-dimethyl-1-yl, 2-butene-2,3-dimethyl-1-yl, 1-butene-2,3-dimethyl-1-yl, 1-butene-2,3-dimethyl-3-yl. The C₂₋₆ alkenyl group may contain one or more substituents and may further be, for example, and without limitation, interrupted by one or more heteroatoms which are independently nitrogen, sulfur or oxygen.

The alkynyl group as used anywhere herein unless otherwise specified, may have from 1 to 30 carbon atoms. In some embodiments the alkyl chain may have, for example, from 1 to 18 carbon atoms. In other embodiments, the alkynyl chain may be, for example, a C₁₋₆ alkynyl, and may be without limitation, any straight or branched alkynyl, for example, 1-ethynyl, 1-propynyl, 2-propynyl, 1-butynyl, 2-butynyl, 3-butynyl, 1-butyn-3-yl, 1-pentynyl, 2-pentyn-1-yl, 3-pentyn-1-yl, 3-pentyn-2-yl, 4-pentyn-1-yl, 1-butyn-3-methyl-1-yl, 1-butyn-3-methyl-4-yl, 1-hexyn-1-yl, 2-hexyn-1-yl, 3-hexyn-1-yl, 4-hexyn-1-yl, 5-hexyn-1-yl, 1-pentyn-4-methyl-1-yl, 1-pentyn-3-methyl-1-yl, 2-pentyn-4-methyl-1-yl, 1-butyn-2,2-dimethyl-1-yl or 3-butyn-2,2-dimethyl-1-yl. The C₂₋₆ alkynyl group may contain one or more substituents and may be, for example, and without limitation, interrupted by one or more heteroatoms which are independently nitrogen, sulfur or oxygen.

The aryl, arylthio, aryloxy as used anywhere herein unless otherwise specified, may have from 6 to 60. In some embodiments the aryl, arylthio, aryloxy may have, for example, from 6 to 30 carbon atoms. The arylamino as used anywhere herein unless otherwise specified, may have from 6 to 180 carbon atoms. In some embodiments, the arylamino may have, for example, from 6 to 120 carbon atoms. The heteroaryl, heteroaryloxy, heteroarylthio as used anywhere herein unless otherwise specified, may have from 3 to 120. In some embodiments the heteroaryl, heteroaryloxy, heteroarylthio may have, for example, from 3 to 60 carbons atoms. The heteroarylamino group as used anywhere herein unless otherwise specified, may have from 3 to 180 carbon atoms. In some embodiments the heteroarylamino may have, for example, from 3 to 120 carbon atoms. For the purpose of illustration, examples of compounds may include, phenyl, phenoxy, pyridinyl and others, as would be known to a person skilled in the art.

For the purpose of illustration and without limitation, an arylene or a heteroarylene are, for example, phenylene, naphthylene, pyridinylene, indenylene, azulenylene or phenanthrenylene.

Compounds of the invention may be prepared using standard procedures known to a person skilled in the art, and by analogy to the more specific procedures set out herein for preparing specific compounds of the invention. The specific procedures described are provided as guidance only and are not intended to be limiting.

Exemplary compounds of the invention, such as compounds 1, 2, 3 or 4, as described previously, may be prepared starting from 2,7-dibromofluorenone (scheme 1), which may be obtained upon chromic acid based oxidation of 2,7-dibromofluorene. Palladium catalyzed based coupling of the dibromoketone with 9,9-dihexyfluorene-2-boronic acid yielded compound A. Bromination of compound A yielded the bright orange compound B, which may be further coupled with 3,5-Bis(trifluoromethyl)phenylboronic acid in a palladium catalyzed reaction to yield compound C. The 9-fluorenone, compounds A or C can be used for the preparation compounds 1, 2, 3 or 4, as shown in scheme 2 or 3.

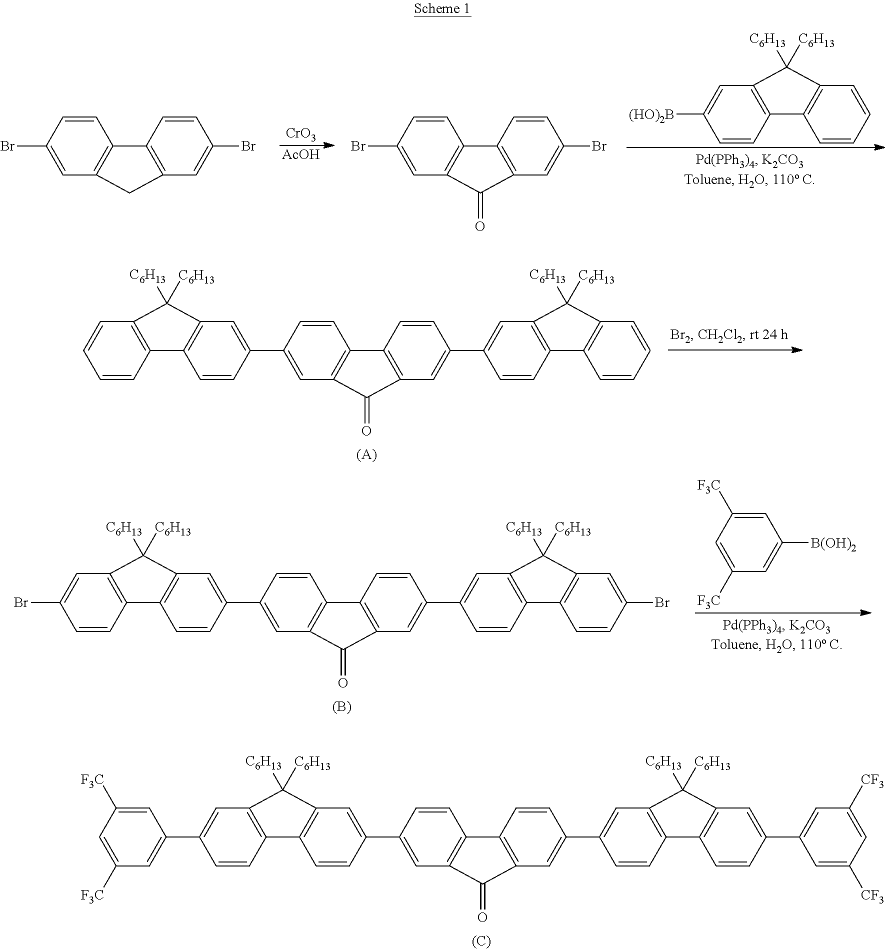

In the preparation of compound 1 (scheme 2), 2,5-dibromothene was reacted with magnesium metal to form a digrignard which was reacted with 9-fluorenone, and was further reduced in the presence of tin (II) dichloride to provide compound 1. Analogously, the 3,5-dibromothiophene was reacted with butyl lithium to a dilithium thiophene, which can be reacted with either compound A or C, in the presence of tin (II) dichloride to produce compounds 2 and 3, respectively.

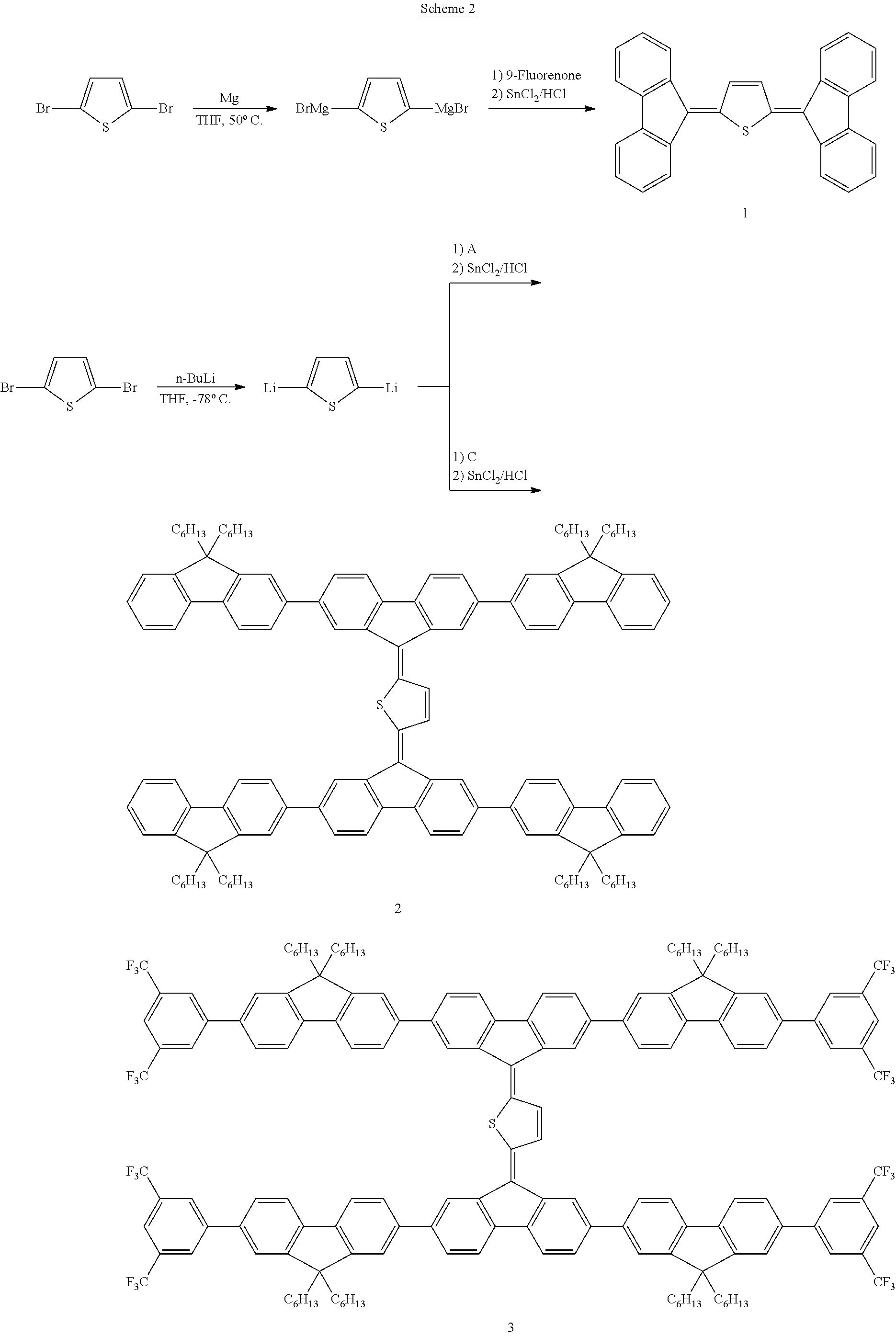

The preparation of compound 4 is shown in scheme 3, which is similar to that of the preparation of compound 1, where a dibromothiophene is used, however, in preparing compound 4, a dibromo-dithiophene was converted into a Grignard reagent and reacted with 9-fluorenone, and then reduced in the presence of tin (II.) dichloride to yield compound 4.

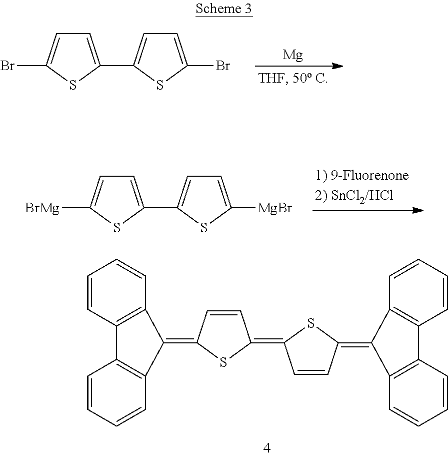

The following may be considered when designing materials of the invention:

1. The rigidity of Ar₁=Qu=Ar₂ may effect charge transport as well as the low bandgap of the materials, usually below 2.0 eV. Low bandgap can be a basic requirement for the materials to potentially have high efficiency in OPV devices.
2. Electron donating groups may increase the hole mobility of the materials.
3. Electron withdrawing groups may stabilize the structure by lowering the HOMO level, and hence also adjusting the HOMO and LUMO level with the desired range, which makes the materials potential good n-type materials for organic electronic devices.

Therefore, such a design may not only provide a low bandgap of the material, but may also increase the charge mobility for TFT application and may adjust HOMO and LUMO levels suitable for most OPV applications.

Materials of the invention could be dissolved in common organic solvent, for example, chloroform, toluene, ethyl benzoate, 1,1,2,2-tetrachloroethane, etc. and could be processed into thin films through spin coating or other thin film preparation method. The thin film of the materials could be used as the active layer or dopant active layer for TFT and OPV devices, for instances, in conjunction with electrodes and dielectric layer, for example.

In an embodiment of the invention, there is provided a semiconductor device having:
a source electrode and a drain electrode separated from a gate electrode by a gate dielectric; and
a semiconductor layer containing a compound of the invention, as described herein, either over or under the source electrode and the drain electrode, to form a charge transport channel.

In another embodiment of the invention, the semiconductor device is a transistor, such as an organic field effect transistor (OFET) or an organic thin film transistor (OTFT); light emitting semiconductor, such as a organic light emitting diode; photoconductor; current limiter; thermister; p-n junction; field-effect diode or Schottky diode.

In a further embodiment of the invention, there is provided an organic thin film transistor device containing:
a plurality of electrically conducting gate electrodes disposed in or on a substrate;
a gate insulator layer disposed in or on the electrically conducting gate electrodes;
an organic semiconductor layer disposed in or on the gate insulator layer substantially overlapping the gate electrodes; and
a plurality of sets of electrically conductive source and drain electrodes disposed in or on the organic semiconductor layer such that each of the sets in alignment with each of the gate electrodes;
   wherein the organic semiconductor layer is a compound of formula (I), (Ia) or (Ib) as described above.

In the above devices and for the purpose of illustration and without limitation, the semiconductor layer may have a thickness of from 10 nm to 10 μm, including any intermediate value or range.

According to a further aspect of the invention, there is provided a process for preparing an organic thin film transistor device containing the steps of:
depositing a plurality of electrically conducting gate electrodes in or on a substrate;
depositing a gate insulator layer in or on the electrically conducting gate electrodes;
depositing a layer of a compound of formula (I), (Ia) or (Ib) in or on the insulator layer such that the layer substantially overlaps the gate electrodes; and
depositing a plurality of sets of electrically conductive source and drain electrodes in or on the layer such that each of the sets is in alignment with each of the gate electrodes;
   thereby producing the organic thin film transistor device.

### EXAMPLES

The following examples are intended as exemplary only and not in any way intended to limit the scope of the present invention.

### Instruments

Nuclear magnetic resonance (NMR) spectra were collected on a Bruker™ DPX 400 M Hz spectrometer using chloroform-d or dichloromethane-d2 as the solvent and tetramethylsilane (TMS) as an internal standard. Matrix-Assisted Laser Desorption/Ionization Time-Of-Flight (MALDI-TOF) mass spectra were obtained on a Bruker™ Autoflex TOF/TOF instrument. Differential scanning calorimetry (DSC) was carried out under nitrogen on a TA Instrument DSC 2920 module (scanning rate of 20° C./min). Thermal gravimetric analysis (TGA) was carried out using a TA Instrument TGA 2050 module (heating rate of 20° C./min). Cyclic voltammetry (CV) experiments were performed on an Autolab potentiostat (model PGSTAT30). All CV measurements were recorded in dichloromethane with 0.1 M tetrabutylammonium hexafluorophosphate as supporting electrolyte (scan rate of 50 mV/s) using a conventional three electrode configuration consisting of a platinum wire working electrode, a gold counter electrode, and a Ag/AgCl in 3 M KCl reference electrode. The measured potentials were converted to saturated calomel electrode (SCE) and the corresponding ionization potential (IP) and electron affinity (EA) values were derived from the onset redox potentials, based on −4.4 eV as the SCE energy level relative to vacuum (EA=Ered-onset+4.4 eV, IP=Eox-onset+4.4 eV). The absorption spectra were recorded on a Shimadzu™ UV-3101 PC UV-vis-NIR spectrophotometer using dichloromethane solution with concentration ranging from 1.8×10⁻⁶ to 5.7×10⁻⁶ M.

### Example 1

### Synthesis of 2,7-dibromofluorenone (scheme 1)

This compound was prepared following the procedure reported in the literature (Tetrahedron 2006, 62, 3355-3361). A mixture of 2,7-dibromofluorene (10.0 g, 30.9 mmol) and CrO₃ (12 g, 0.12 mol) suspended in 250 mL acetic acid, and stirred at room temperature for 12 h. The resulting yellow precipitate was collected by suction filtration, washed with water thoroughly, and dried under vacuum to provide the product as yellow solid (10.1 g, 98% yield). ¹H NMR (400 MHz, CDCl₃) 7.77 (s, 2H), 7.63 (d, 2H, J=8.0 Hz), 7.39 (d, 2H, J=8.0 Hz).

### Synthesis of Compound A (Scheme 1)

To a mixture of 2,7-dibromofluorenone (1.8 g, 5.3 mmol), prepared as above, 9,9-dihexyfluorene-2-boronic acid (5.0 g, 13.2 mmol), and tetrakis(triphenylphosphine)palladium (0.123 g, 0.053 mmol, 1% per C—Br bond), was added degassed K₂CO₃ aqueous (20 mL) and degassed toluene (50 mL). The solution was refluxed under N₂ protection for 24 h. The resulting brown solution was extracted with CH₂Cl₂ (50 mL×4). The combined organic layers were dried over MgSO₄ and evaporated under reduced pressure to remove the solvent. The residue was then purified with a silicon gel column using CH₂Cl₂/hexane (1:4.5) as the eluent to obtain the desired product as a yellow solid (4.26 g, 95% yield). ¹H NMR (400 MHz, CDCl₃): δ (ppm) 8.03 (d, 2H, J=0.8 Hz), 7.83 (dd, 2H, J=7.6, 1.2 Hz), 7.78 (dd, 2H, J=7.6 Hz), 7.74 (dd, 2H, J=7.6, 1.2 Hz), 7.60-7.66 (m, 6H), 7.31-7.37 (m, 6H), 2.02 (t, 8H, J=8.0 Hz), 1.06-1.16 (m, 24H), 0.76 (t, 12H, J=7.2 Hz), 0.66 (hexa, 8H, J=6.8 Hz). MS (MALDI): m/z=844.71 (calcd. for C₆₃H₇₂O: 844.57).

### Synthesis of Compound B (Scheme 1)

To a CH₂Cl₂ (20 mL) solution of compound A (1.50 g, 1.78 mmol), prepared as above, at 0° C., was slowly added a CH₂Cl₂ solution (10 mL) of Br₂ (0.60 g, 3.75 mmol). After being stirred for 30 min, the solution was warmed to ambient temperature by removing the ice bath, and was stirred at this temperature for 24 h. Subsequently an aqueous solution of sodium sulfite (10 mL , 1M) was then added to consume the excess bromine. The resulting yellow-orange solution was extracted with CH₂Cl₂ (30 mL×4), and the organic phase was combined, washed with brine (20 mL×3), dried over MgSO₄. The solvent was then removed under reduced pressure to afford compound B as bright orange solid (1.75 g, 98% yield). ¹H NMR (400 MHz, CDCl₃): δ (ppm) 8.02 (d, 2H, J=1.2 Hz), 7.82 (dd, 2H, J=7.6, 1.2 Hz), 7.75 (d, 2H, J=8.0 Hz), 7.58-7.66 (m, 8H), 7.47-7.49 (m, 4H), 1.96-2.05 (m, 8H), 1.05-1.15 (m, 24H), 0.78 (t, 12H, J=7.2 Hz), 0.62-0.69 (m, 8H).

### Synthesis of Compound C (Scheme 1)

To a mixture of compound B (1.50 g, 1.50 mmol), prepared as above, 3,5-Bis(trifluoromethyl)phenylboronic acid (0.89 g, 3.45 mmol), and tetrakis(triphenylphosphine)palladium (0.070 g, 0.061 mmol, 2% per C—Br bond), was added degassed K₂CO₃ aqueous (20 mL) and degassed toluene (50 mL). The solution was refluxed under N₂ protection for 24 h. The resulting brown solution was extracted with CH₂Cl₂ (50 mL×4). The combined organic layers were dried over MgSO₄ and evaporated under reduced pressure to remove the solvent. The residue was then purified with a silicon gel column using CH₂Cl₂/hexane (1:5) as the eluent to obtain the desired product as an orange solid (1.21 g, 64% yield). ¹H NMR (400 MHz, CDCl₃): δ (ppm) 8.08 (s, 4H), 8.05 (s, 2H), 7.83-7.87 (m, 8H), 7.60-7.69 (m, 8H), 7.56 (s, 2H), 2.11 (t, 8H, J=8.0 Hz), 1.05-1.15 (m, 24H), 0.69-0.78 (m, 20H). MS (MALDI): m/z=1256.86 (calcd. for C₇₈H₇₆F₁₂O: 1256.58).

### Example 2

### Synthesis of Compound 1 (Scheme 2)

To a mixture of 2,5-dibromothiophene (1.00 g, 4.13 mmol) and metal Mg (0.218 g, 9.1 mmol) was added 40 mL of freshly distilled anhydrous THF. After being stirred for 3 h at 50° C., the solution was cooled down to 0° C. with ice bath, and a THF solution (15 mL) of 9-fluorenone (1.56 g, 8.67 mmol) was added dropwise. The solution was then stirred at this temperature for 30 min before being warmed up to ambient temperature by removing the ice bath. After being stirred for another hour, a saturated SnCl₂ solution in 10% hydrochloric acid (40 mL) was added dropwise. The color of the solution immediately turned to deep red. The solution was stirred at ambient temperature for two more hours, and then was extracted with CH₂Cl₂ (100 mL×4). The organic phase was combined and dried over MgSO₄. The organic solvent was then removed by reduced pressure. Subsequent recrystallization using CH₂Cl₂/n-hexane as the solvent afforded compound 1 as dark green needle crystals (0.625 g, 37% yield). ¹H NMR (400 MHz, CD₂Cl₂): δ (ppm) 8.30 (s, 2H), 8.27 (d, 2H, J=8.0 Hz), 8.07-8.09 (m, 2H), 7.80-7.84 (m, 4H), 7.51 (t, 2H, J=7.2 Hz), 7.44 (t, 2H, J=7.6 Hz), 7.38-7.40 (m, 4H). MS (MALDI): m/z=410.12 (calcd. for C₃₀H₁₈S: 410.21). HOMO: −5.38 eV, LUMO: −3.52 eV.

### Synthesis of Compound 2 (Scheme 2)

2,5-Dibromothiophene (0.10 g, 0.41 mmol), as prepared above, was dissolved in 10 mL of freshly distilled anhydrous THF, and was cooled to −78° C. with acetone/dry ice bath. To this solution was added n-hexane solution of butyllithium (1.6 M, 0.54 mL, 0.86 mmol) slowly via a syringe. The solution was then stirred at this temperature for 1 h before a THF solution (10 mL) of compound A (0.77 g, 0.91 mmol) was added dropwise. The solution was subsequently stirred at this temperature for 30 min before acetone/dry ice bath was removed. After another stirring at ambient temperature for 2 h, a saturated SnCl₂ solution in 10% hydrochloric acid (20 mL) was added dropwise. The color of the solution turned to deep rose red immediately. The solution was stirred for another 2 h, and extracted with CH₂Cl₂ (50 mL×3). The organic phase was combined, dried over MgSO₄, and evaporated under reduced pressure to remove the solvent. The residue was subsequently purified with alumina (neutral) column using CH₂Cl₂/n-hexane (1:5) as the eluent. The deep rose red band was collected to obtain compound 2 as deep purple solid (0.230 g, 32% yield). ¹H NMR (400 MHz, CD₂Cl₂): δ (ppm) 8.63 (s, 2H), 8.54 (s, 2H), 8.41 (s, 2H), 7.95 (dd, 4H, J=8.0, 2.4 Hz), 7.87-7.90 (m, 4H), 7.71-7.82 (m, 12H), 7.66 (d, 2H, J=7.6 Hz), 7.32-7.45 (m, 10H), 1.97-2.15 (m, 16H), 0.94-1.16 (m, 48H), 0.69-0.79 (m, 40H). MS (MALDI): m/z=1739.05 (calcd. for C₁₃₀H₁₄₆S: 1739.22). HOMO: −5.43 eV, LUMO: −3.59 eV.

### Synthesis of Compound 3 (Scheme 2)

2,5-Dibromothiophene (0.05 g, 0.21 mmol), as prepared above, was dissolved in 10 mL of freshly distilled anhydrous THF, and was cooled to −78° C. with acetone/dry ice bath. To this solution was added n-hexane solution of butyllithium (1.6 M, 0.27 mL, 0.43 mmol) slowly via a syringe. The solution was then stirred at this temperature for 1 h before a THF solution (10 mL) of compound C (0.57 g, 0.91 mmol) was added dropwise. The solution was subsequently stirred at this temperature for 30 min before acetone/dry ice bath was removed. After another stirring at ambient temperature for 2 h, a saturated SnCl₂ solution in 10% hydrochloric acid. (20 mL) was added dropwise. The color of the solution turned to deep rose red immediately. The solution was stirred for another 2 h, and extracted with CH₂Cl₂ (50 mL×3). The organic phase was combined, dried over MgSO₄, and evaporated under reduced pressure to remove the solvent. The residue was subsequently purified with alumina (neutral) column using CH₂Cl₂/n-hexane (1:6) as the eluent. The deep rose red band was collected to obtain compound 3 as deep purple solid (0.198 g, 37% yield). ¹H NMR (400 MHz, CD₂Cl₂): δ (ppm) 8.65 (s, 2H), 8.55 (s, 2H), 8.42 (s, 2H), 8.17 (s, 4H), 7.64-8.00 (m, 36H), 7.28-7.32 (m, 4H), 2.04-2.20 (m, 16H), 1.00-1.14 (m, 48H), 0.67-0.79 (m, 40H). MS (MALDI): m/z=2587.14 (calcd. for C₁₆₂H₁₅₄F₂₄S: 2587.25). HOMO: −5.44 eV, LUMO: −3.59 eV.

### Example 3

### Synthesis of Compound 4 (Scheme 3)

To a mixture of 2,5-dibromothiophene (0.50 g, 1.54 mmol) and metal Mg (0.089 g, 3.71 mmol) was added 20 mL of freshly distilled anhydrous THF. After being stirred for 3 h at 50° C., the solution was cooled down to ambient temperature. To another flask containing 9-fluorenone (0.61 g, 3.39 mmol) in 10 mL anhydrous THF solution that was cooled to 0° C., was added the clear solution of the resulting Grignard reagent via a double needle. The mixed solution was then stirred at this temperature for 30 min before being warmed up to ambient temperature by removing the ice bath. After being stirred for another hour, a saturated SnCl₂ solution in 10% hydrochloric acid (40 mL) was added dropwise. The color of the solution immediately turned to deep blue. The solution was stirred at ambient temperature for two more hours, and the deep blue precipitate was collected by suction filtration and washed with THF, water, methanol and CH₂Cl₂ to afford compound 4 as dark blue solid (0.125 g, 16% yield). The solubility of compound 4 is very poor in any solvent, and NMR spectrum was not available. MS (MALDI): m/z=492.14 (calcd. for C₃₄H₂₀S₂: 492.29). HOMO: −4.98 eV, LUMO: −3.70 eV.

### Example 4

### TFT Device Fabrication and Measurement

Top contact bottom gate Organic Thin Film Transistors (OTFT) shown in FIG. 2 were fabricated using the following steps:

### A. Substrate Preparation:

1. p⁺-Si (or n⁺-Si)/SiO₂ substrates are used for OTFT fabrication where p⁺-Si (or n⁺—Si) and SiO₂ works as gate contact and gate dielectric respectively.
2. Substrates as stated above were subjected to cleaning using ultrasonication in acetone, methanol and de-ionized water.
3. Cleaned wafers were dried under flow of nitrogen and heated at 100° C. for 5 minutes.
4. The cleaned and dried p⁺-Si (or n⁺-Si)/SiO₂ wafers were subjected to UV-ozone treatment for 20 minutes.
B. Self-Assembled Monolayer (SAM) Grown p⁺-Si (or n⁺-Si)/SiO₂ Substrates:

In order to improve organization of organic molecules on p⁺-Si (or n⁺-Si)/SiO₂ substrates, few substrates were used after self-assembled monolayer (SAM) treatment.

5. Hexa-methyldichlorosilane (HMDS) SAM treatment: p⁺-Si (or n⁺-Si)/SiO₂ substrates were exposed to HMDS vapours at room temperature inside a glove box under N₂ gas overnight.
6. Octyltricholorosilane (OTS) SAM treatment: p⁺-Si (or n⁺-Si)/SiO₂ substrates were kept in a dessicator with a few drops of OTS. The dessicator was first placed under vacuum for 3 minutes and then subsequently placed in an oven at 110° C. for three hours. After that p⁺-Si (or n⁺-Si)/SiO₂ substrates were removed from the dessicator, thoroughly rinsed with isopropanol and dried under flow of nitrogen gas.

### C. Organic Thin Film Growth:

A thin film of compound 1 was grown on above stated substrate using thermal evaporation technique. 35-40 nm thick film of compound 1 was grown at vacuum of ˜10⁻⁶ mbar.

### D. OTFT Fabrication:

Once organic thin films were grown on substrates, top contact bottom gate OTFTs were fabricated by depositing ˜100 nm of gold as source and drain contacts using shadow masks. The typical OTFT devices had 50, 100 and 200 μm channel length (L) in combination to 1 mm and 3 mm channel width (W).

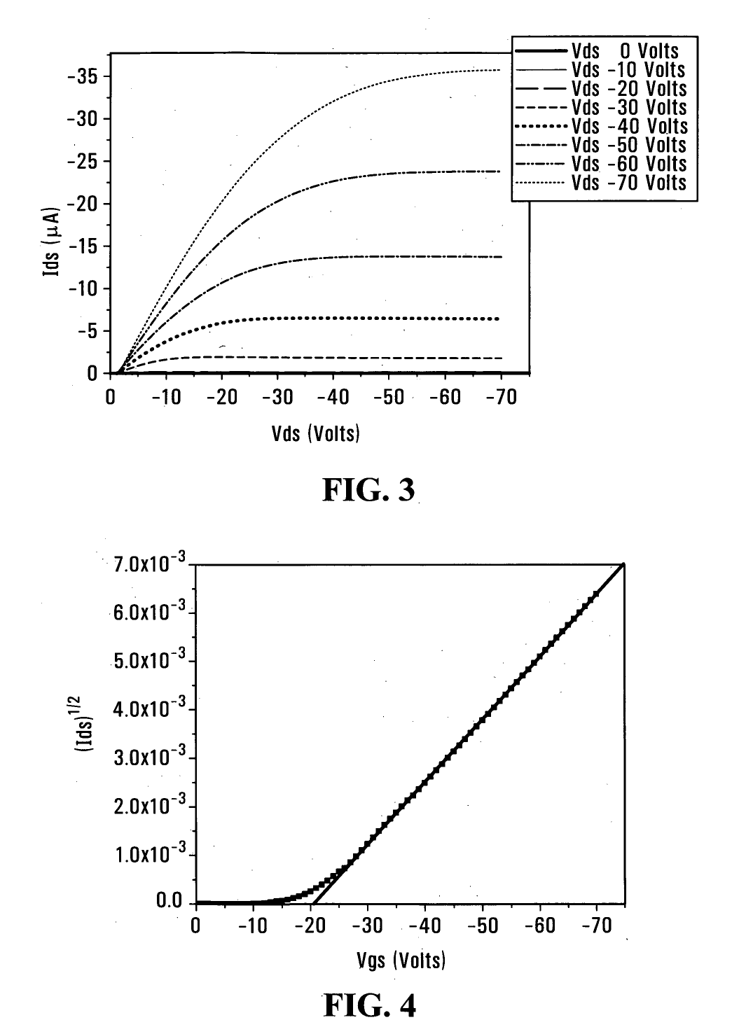

The above fabricated OTFTs were characterized in glove box under nitrogen using Keithley™ 4200 parameter analyzer. Typical V_{ds}-I_{ds} and V_{gs}-I_{ds} characteristic of top contact OTFTs using QD31 as organic active layer on OTS treated p⁺-Si/SiO₂ substrates having L/W (100/3000) are shown in FIG. 3 and FIG. 4, respectively. Compound 1 based OTFTs have shown p-type charge transport exhibiting saturation hole mobility ˜0.06 cm²/V-sec and threshold voltage (V_{T}) ˜20 volts.

### Example 5

### OPV Device Fabrication and Measurement

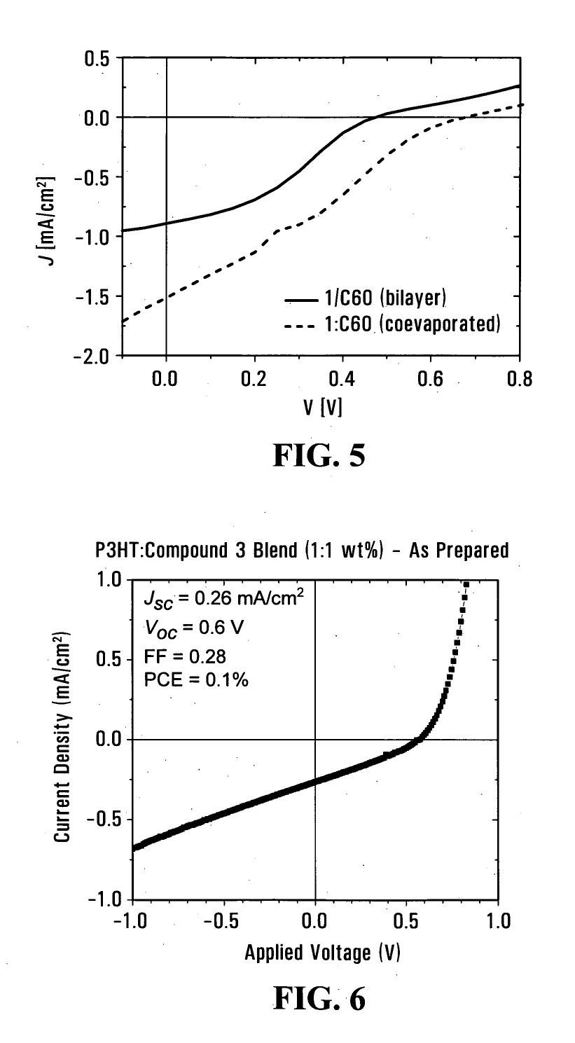

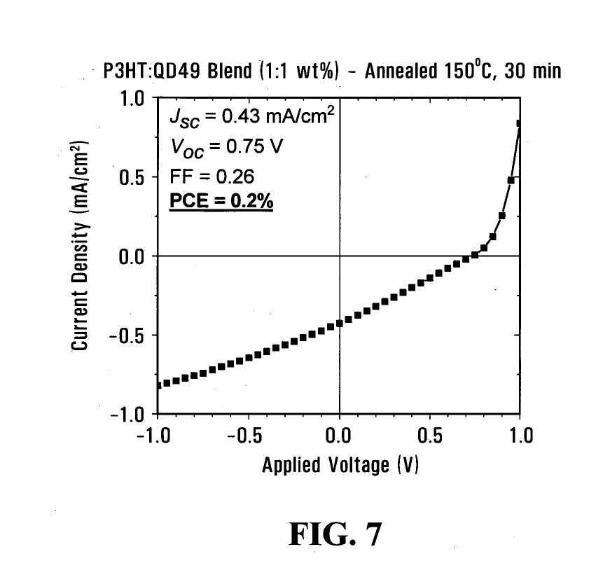

Organic photovoltaic (OPV) devices using compound 1 as the donor and C₆₀ as the acceptor were fabricated. Both compound 1 and C₆₀ have been evaporated on top of the glass/ITO/PEDOT:PSS substrates. Two configuration, namely ITO/PEDOT:PSS/1/C₆₀/Al (bilayer) and ITO/PEDOT:PSS/1/1:C₆₀/C₆₀/Al (co-evaporation) were investigated. The performance of those OPV devices after post-annealing are shown in FIG. 5. The complete solar cell parameters are listed in Table 1. Organic photovoltaic (OPV) devices using compound 3 as the acceptor and P3HT as the donor were also fabricated. OPV device with a structure of ITO/PEDOT:PSS/P3HT/3/Al was fabricated through spin coating of the solution of P3HT and compound 3 (1:1 wt %) dissolved in toluene. The performance of the solution processed OPV devices (as prepared) are shown in FIG. 6. The complete solar cell parameters are listed in Table 1.

As shown in FIG. 5, OPV device with a structure of ITO/PEDOT:PSS/1/1:C₆₀/C₆₀/A1 shows a higher open circuit voltage (V_{oc}) and short-circuit current density (J_{sc}) compared to the one with a configuration of ITO/PEDOT:PSS/1/C₆₀/Al. The power conversion efficiency (PCE) is 0.60% and 0.30% for the co-evaporated and bilayer structure, respectively. The better performance of the co-evaporated sample compared to the bilayer one is attributed to the possibility of easier exciton dissociation and better charge transport in the co-evaporated sample.

TABLE 1
Solar Cell Characteristics Parameters: Open-Circuit Voltage
(V_{oc}), Short-Circuit Current Density (J_{sc}), Fill Factor
(FF), and PCE (η) for the prepared devices.

| Sample | V_{oc} [V] | J_{sc} [mA/cm²] | FF | η [%] |
|---|---|---|---|---|
| Compound 1/C60 | 0.48 | 0.89 | 0.35 | 0.30 |
| (bilayer) | | | | |
| Compound 1: C60 | 0.68 | 1.52 | 0.27 | 0.60 |
| (co-evaporation) | | | | |
| P3HT/Compound 3 | 0.60 | 0.26 | 0.28 | 0.10 |
| (As-prepared) | | | | |
| P3HT/Compound 3 | 0.75 | 0.43 | 0.26 | 0.20 |
| (thermal annealled) | | | | |

1. A compound of formula (I):
Ar₁=(Qu)ₘ=Ar₂ (I)
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen, and is optionally substituted by one or more substantially planar conjugated aromatic structures independently chosen, and
m is from 1 to 20;
with the proviso that the compound of formula (I) is not a compound of formula (1)

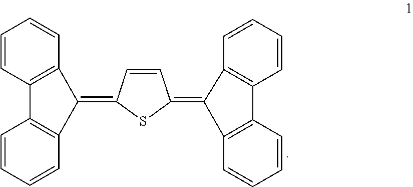

2. A compound of formula (Ib) 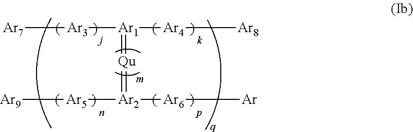
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen,
Ar₃, Ar₄, Ar₅, Ar₆, Ar₇, Ar₈, Ar₉ and Ar₁₀ each independently is a substituted or unsubstituted, substantially planar conjugated aromatic structure having from 5 to 50 carbon atoms, and optionally containing one or more heteroatoms independently chosen,
m is from 1 to 20, and
j, k, n, p each, independently, is from 0 to 20,
q is 1 or more.

3. The compound of claim 1, wherein the substantially planar conjugated aromatic structure forms a rigid substantially planar conjugated aromatic structure.

4. The compound of claim 1, wherein the substantially planar conjugated aromatic structure forms a bridged substantially planar conjugated aromatic structure.

5. The compound of claim 1 having a bandgap of <2.0 eV.

6. The compound of claim 1, wherein =Qu= is 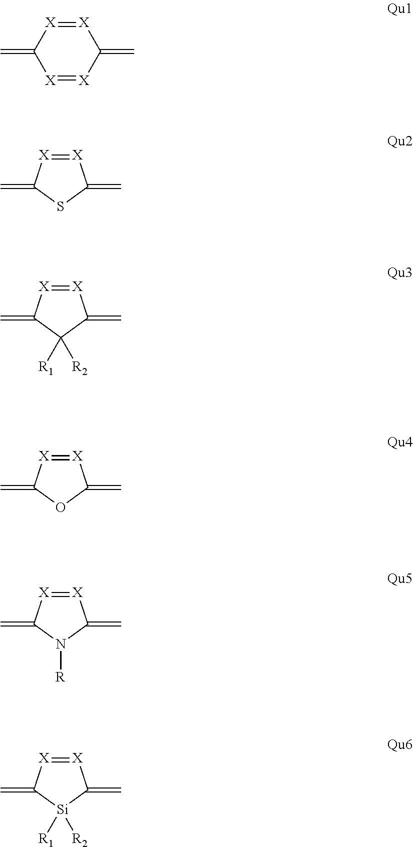
where each X, independently, is CH, N or P, and optionally contains, one or more substitutents.

7. The compound of claim 1, wherein the substitutions on Ar₁ to Ar₁₀, may be one or more electron donating groups, electron withdrawing groups or a combination thereof.

8. A compound of formula 2, 3 or 4

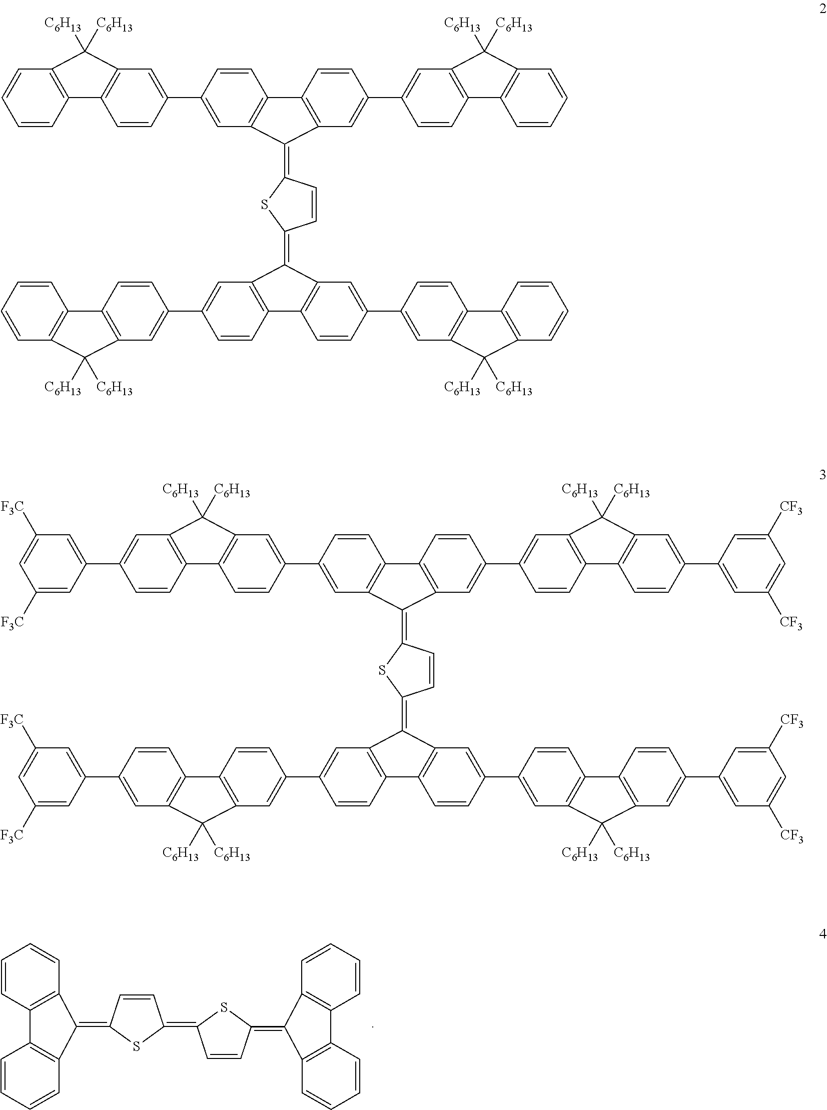

9. A semiconductor device having a semi-conductor layer containing the compound of formula (I) or formula (Ib)
wherein Ar₁=(Qu)ₘ=Ar₂ (I),
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen, and is optionally substituted by one or more substantially planar conjugated aromatic structures independently chosen, and
m is from 1 to 20;
and wherein 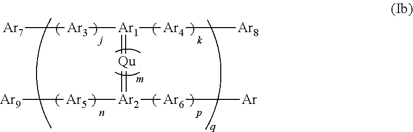
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen,
Ar₃, Ar₄, Ar₅, Ar₆, Ar₇, Ar₈, Ar₉ and Ar₁₀ each independently is a substituted or unsubstituted, substantially planar conjugated aromatic structure having from 5 to 50 carbon atoms, and optionally containing one or more heteroatoms independently chosen,
m is from 1 to 20, and
j, k, n, p each, independently, is from 0 to 20,
q is 1 or more.

10. A semiconductor device having:
a source electrode and a drain electrode separated from a gate electrode by a gate dielectric; and
a semiconductor layer having the compound of formula (I) or formula (Ib) either over or under the source electrode and the drain electrode to form a charge transport channel,
wherein
Ar₁=(Qu)ₘ=Ar₂ (I)
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen, and is optionally substituted by one or more substantially planar conjugated aromatic structures independently chosen, and
m is from 1 to 20;
and wherein 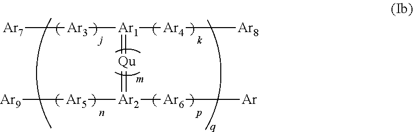
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen,
Ar₃, Ar₄, Ar₅, Ar₆, Ar₇, Ar₈, Ar₉ and Ar₁₀ each independently is a substituted or unsubstituted, substantially planar conjugated aromatic structure having from 5 to 50 carbon atoms, and optionally containing one or more heteroatoms independently chosen,
m is from 1 to 20, and
j, k, n, p each, independently, is from 0 to 20,
q is 1 or more.

11. The semiconductor device of claim 10, wherein the device is a transistor, light emitting semiconductor, photoconductor, current limiter, thermister, p-n junction, field-effect diode or Schottky diode.

12. An organic thin film transistor device containing:
a plurality of electrically conducting gate electrodes disposed in or on a substrate;
a gate insulator layer disposed in or on the electrically conducting gate electrodes;
an organic semiconductor layer disposed in or on the gate insulator layer substantially overlapping the gate electrodes; and
a plurality of sets of electrically conductive source and drain electrodes disposed in or on the organic semiconductor layer such that each of the sets in alignment with each of the gate electrodes;
wherein the organic semiconductor layer is the compound of formula (I) or formula (Ib),
wherein Ar₁=(Qu)ₘ=Ar₂ (I),
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen, and is optionally substituted by one or more substantially planar conjugated aromatic structures independently chosen, and
m is from 1 to 20;
and wherein 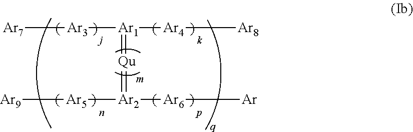
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen,
Ar₃, Ar₄, Ar₅, Ar₆, Ar₇, Ar₈, Ar₉ and Ar₁₀ each independently is a substituted or unsubstituted, substantially planar conjugated aromatic structure having from 5 to 50 carbon atoms, and optionally containing one or more heteroatoms independently chosen,
m is from 1 to 20, and
j, k, n, p each, independently, is from 0 to 20,
q is 1 or more.

13. A process for preparing an organic thin film transistor device containing the steps of:
depositing a plurality of electrically conducting gate electrodes in or on a substrate;
depositing a gate insulator layer in or on the electrically conducting gate electrodes;
depositing a layer of the compound of formula (I) or formula (Ib) in or on the insulator layer such that the layer substantially overlaps the gate electrodes; and
depositing a plurality of sets of electrically conductive source and drain electrodes in or on the layer such that each of the sets is in alignment with each of the gate electrodes;
thereby producing the organic thin film transistor device,
wherein Ar₁=(Qu)ₘ=Ar₂ (I)
where
each Qu is independently a substituted or unsubstituted, substantially planar 5 to 8 membered conjugated ring, optionally fused to one or more aromatic moieties independently chosen, and may contain one or more heteroatoms independently chosen, Ar₁ and Ar₂ each independently is a substituted or unsubstituted, substantially planar conjugated aromatic structure having from 5 to 50 carbon atoms, optionally having one or more heteroatoms independently chosen, and is optionally substituted by one or more substantially planar conjugated aromatic structures independently chosen, and
m is from 1 to 20;
and wherein 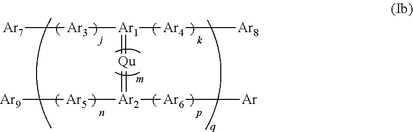
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen,
Ar₃, Ar₄, Ar₅, Ar₆, Ar₇, Ar₈, Ar₉ and Ar₁₀ each independently is a substituted or unsubstituted, substantially planar conjugated aromatic structure having from 5 to 50 carbon atoms, and optionally containing one or more heteroatoms independently chosen,
m is from 1 to 20, and
j, k, n, p each, independently, is from 0 to 20,
q is 1 or more.

14. An organic thin film transistor having an active layer containing the compound of formula (I) or formula (Ib)
wherein Ar₁=(Qu)ₘ=Ar₂ (I)
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen, and is optionally substituted by one or more substantially planar conjugated aromatic structures independently chosen, and
m is from 1 to 20;
and wherein 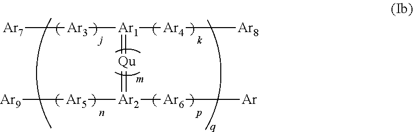
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen,
Ar₃, Ar₄, Ar₅, Ar₆, Ar₇, Ar₈, Ar₉ and Ar₁₀ each independently is a substituted or unsubstituted, substantially planar conjugated aromatic structure having from 5 to 50 carbon atoms, and optionally containing one or more heteroatoms independently chosen,
m is from 1 to 20, and
j, k, n, p each, independently, is from 0 to 20,
q is 1 or more.

15. A photovoltaic cell having an active layer containing the compound of formula (I) or formula (Ib),
wherein Ar₁=(Qu)ₘ=Ar₂ (I)
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen, and is optionally substituted by one or more substantially planar conjugated aromatic structures independently chosen, and
m is from 1 to 20;
and wherein 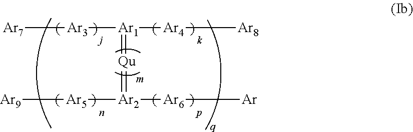
where
each Qu is independently a substituted or unsubstituted, substantially planar 5 to 8 membered conjugated ring, optionally fused to one or more aromatic moieties independently chosen, and may contain one or more heteroatoms independently chosen,
Ar₁ and Ar₂ each independently is a substituted or unsubstituted, substantially planar conjugated aromatic structure having from 5 to 50 carbon atoms, optionally having one or more heteroatoms independently chosen,
Ar₃, Ar₄, Ar₅, Ar₆, Ar₇, Ar₈, Ar₉ and Ar₁₀ each independently is a substituted or unsubstituted, substantially planar conjugated aromatic structure having from 5 to 50 carbon atoms, and optionally containing one or more heteroatoms independently chosen,
m is from 1 to 20, and
j, k, n, p each, independently, is from 0 to 20,
q is 1 or more.

16. The compound of claim 2, wherein the substantially planar conjugated aromatic structure forms a rigid substantially planar conjugated aromatic structure.

17. The compound of claim 2, wherein the substantially planar conjugated aromatic structure forms a bridged substantially planar conjugated aromatic structure.

18. The compound of claim 2, having a bandgap of <2.0 eV.

19. The compound of claim 2, wherein =Qu= is 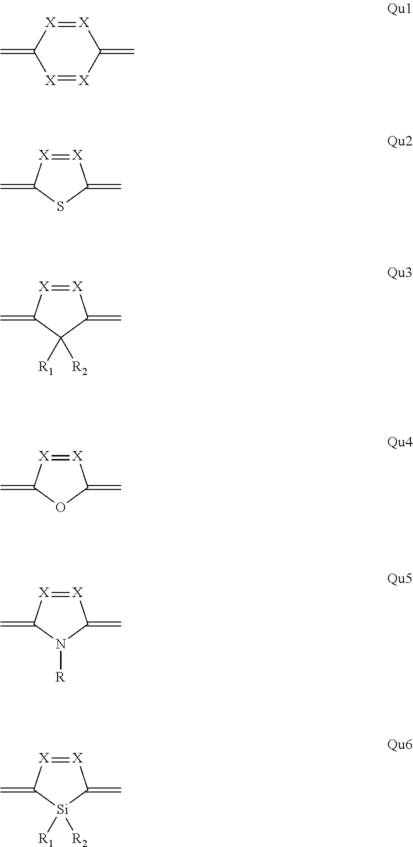
where each X, independently, is CH, N or P, and optionally contains, one or more substitutents.

20. The compound of claim 2, wherein the substitutions on Ar₁ to Ar₁₀, may be one or more electron donating groups, electron withdrawing groups or a combination thereof.
